(19) United States
(12) Patent Application Publication
(10) Pub. No.: US 2020/0337713 A1
(43) Pub. Date: Oct. 29, 2020

(54) Method of Performing Restoration of Knee Surgery

(71) Applicants: Hem Sharad Oza, Ahmedabad (IN); Mita Sharad Oza, Ahmedabad (IN)

(72) Inventors: Sharad Ghanshyam Oza, Ahmedabad (IN); Mita Sharad Oza, Ahmedabad (IN); Hem Sharad Oza, Ahmedabad (IN)

(21) Appl. No.: 16/396,647

(22) Filed: Apr. 27, 2019

(51) Int. Cl. A61B 17/15

(52) U.S. Cl. CPC A61B 17/151; A61B 2017/565; A61B 17/155; A61B 17/157

(57) ABSTRACT

A method of performing a surgery on an arthritic knee by preservation and restoration of the knee joint anatomy. The present invention is not a full open surgery and all the natural protective mechanisms of the knee like muscles, ligaments, cartilage etc. are preserved. The primary objective of the surgical technique is to restore the knee joint anatomy by performing partial cuts to tibia and femur. Restoring the joint space allows natural mechanism of joint movement.

### FIELD OF THE INVENTION

The present invention relates to the method of surgery on a knee joint of a patient and more particularly, it relates to a surgical techniques used to perform restoration of a knee joint to its anatomical condition.

### BACKGROUND OF THE INVENTION

Arthritis of the knees can wear away and destroy the surfaces of the knee joint and ultimately lead to conditions that require a knee replacement. Osteoarthritis (OA or degenerative arthritis) is the most common joint disorder known. Osteoarthritis is characterized by cartilage loss at the joint, and symptoms generally include pain and stiffness. The disease can affect all joints of the body, including the hip, shoulder, ankle, and spine, to name a few. Osteoarthritis of the knee is a common and rapidly growing problem. In osteoarthritis, which is usually caused by old age, angular deformity, or old fractures, the surfaces of the knee gradually wear out. Likewise systemic arthritis affects the synovial or membrane tissue in the joint destroying the surface of the joint. In both osteoarthritis and rheumatoid arthritis, when the surface of the joint is worn away, walking and other activities become difficult and painful.

In the knee joint, the articular cartilage tissue forms a lining, which faces the joint cavity on one side and is linked to the subchondral bone plate by a narrow layer of calcified cartilage tissue on the other side. Articular cartilage is composed of chondrocytes, type II collagen fibril meshwork, proteoglycans and water. Active chondrocytes are unique in that they have a relatively low turnover rate and are sparsely distributed within the surrounding matrix. The collagens give the tissue its form and tensile strength and the interaction of proteoglycans with water give the tissue its stiffness to compression, resilience and durability. Cancellous bone is a less dense structure than that of cortical bone and is also comprised of triple helix strands of collagen fiber, reinforced with hydroxyapatite. The cancellous bone includes void areas with the collagen fiber component contributing in part to torsional and tensile strength.

If the lining becomes worn or damaged, resulting in lesions, joint movement may be painful or severely restricted. Whereas damaged bone typically can regenerate successfully, hyaline cartilage regeneration is quite limited because of its limited regenerative and reparative abilities. Articular cartilage lesions are frequently associated with disability and with symptoms such as joint pain, locking phenomena and reduced or disturbed function. These lesions are difficult to treat because of the distinctive structure and function of hyaline cartilage. Such lesions are believed to progress to severe forms of osteoarthritis. There are many current therapeutic methods being used. None of these therapies has resulted in the successful regeneration of hyaline-like tissue that withstands normal joint loading and activity over prolonged periods.

Currently, the techniques most widely utilized clinically for cartilage defects and degeneration is not articular cartilage substitution procedures, but rather lavage, arthroscopic debridement, and repair stimulation. The main goal of treatments is to reduce or eliminate pain, and restore normal joint function. Both non-surgical and surgical treatments are currently available for this purpose, with the appropriate treatment being selected based in part on the stage and/or severity of the disease. However, surgical intervention becomes necessary, when non-surgical treatments prove ineffective, or for patients with severe pain or bone injury. One type of surgical treatment focuses on unloading forces from the damaged joint. Another type of surgical treatment aims to replace, either partially or wholly, the damaged area of the joint. The choice of procedure depends on the patient's age and activity expectations, the severity of the disease, and the number of knee compartments involved. For example, one surgical treatment seeks to replace the damaged or worn cartilage by way of cartilage resurfacing or cartilage replacement. Other surgical treatments, such as high tibial osteotomy (HTO) or total knee arthroplasty (TKA), are often recommended for patients with severe pain associated with osteoarthritis, especially when other non-invasive options have failed. Both procedures have been shown to be effective in treating knee pain associated with osteoarthritis, at least in the short term. In essence, knee osteotomies adjust the geometry of the knee joint so as to transfer weight-bearing load from arthritic portions of the joint to relatively unaffected portions of the joint. Such procedures are shown in U.S. Pat. Nos. 5,980,526; 6,796,986; 5,911,724; 5,053,039; 5,540,695, and 5,601,565. E.P. Patent Number 2,086,434 comprises a novel method and apparatus for performing an open wedge, high tibial osteotomy. The disclosed invention comprises the provision and use of a novel method and apparatus for forming an appropriate osteotomy cut into the upper portion of the tibia, manipulating the tibia so as to open an appropriate wedge-like opening in the tibia, and then mounting an appropriately-shaped implant at the wedge-like opening in the tibia, so as to stabilize the tibia with the desired orientation, whereby to reorient the lower portion of the tibia relative to the tibial plateau and hence adjust the manner in which load is transferred from the femur to the tibia.

Today all new methods and advanced technologies introduced into medicine have made surgery the most preferred option on the management of arthritis affected joints. It has a proven efficiency also in the treatment of osteoarthritis (OA) of the knee joints. At present, the artificial total knee joint replacement in the skill soft tissue balance is a key step.

U.S. Pat. No. 1,673,991 discloses a total knee arthroplasty (TKA) set that includes a universal cutting jig for shaping the distal femur is disclosed. The universal cutting jig may be used to create a shaped femur that can be receiving multiple and different TKA implant designs. A universal high flexion knee system adapted for left-right use is also disclosed.

J.P. Patent Number 6,055,825 discloses an instrument and method are provided for total knee arthroplasty (TKA). The instrument separates a patient's tibia and femur, in both extension and flexion, to measure a gap and an angle there between. The instrument includes various modular accessories that provide flexibility of usage throughout the TKA procedure and that accommodate different surgical philosophies.

A universal mini invasive cutting guide is described in E.P. Patent Number 2,092,899, that is to be utilized for femoral and tibial resections during total knee arthroplasty, both for left or right limb to be used in conjunction with a computer assisted surgery system (CAS). The guide has a curvature similar to a generic proximal tibia and distal femur.

U.S. Pat. No. 7,458,933 describes a method of performing knee-joint surgery includes positioning the tibia and the femur such that the knee joint is disposed in a bent position.

U.S. Pat. No. 6,379,385 discloses an implant base body of spongiest bone material into which a load carrying support element is embedded. The support element can take the shape of a diagonal cross or a plurality of cylindrical pins.

U.S. Pat. No. 6,096,081 shows a bone dowel with a cortical end cap or caps at ends, a brittle cancellous body and a through-going bore. The use of implants for cartilage defects is much more limited. Aside from the fresh allograft implants and autologous implants, U.S. Pat. No. 6,110,209 shows the use of an autologous articular cartilage cancellous bone paste to fill arthritic defects. Concerns associated with this method are harvest site morbidity and availability, similar to the mosaicplasty method and retention of the implant in the prepared cartilage defect space.

The degenerative joint disease is a serious medical condition. The degenerative changes follow an incessantly progressive course after their onset. Patient begins to seek surgical treatment when they run out of options to efficiently manage the conditions by conservative therapeutic modalities. These above known surgical procedures have high chance of infection, complication, implant failure, durability. While these surgical procedures are effective for their intended purpose, there remains a need for improvement in the relevant art for treating focal defects in articular cartilage in a minimally invasive manner to restore the knee joint. The present invention is “A natural way of treating osteoarthritis of knee to restore the knee joint to its anatomical condition.”

### SUMMARY OF THE INVENTION

The main object of the present invention is to preserve and restore the knee joint as close as anatomically possible. The present invention is a method of treating an arthritic knee by Preservation and Restoration of the Knee joint anatomy. The present invention is not a full open surgery and all the natural protective mechanisms of the knee like muscles, ligaments, cartilage etc. are preserved. Due to this reason the post-operative physiotherapy for muscle strength is more effective in gaining mobility and power in a patient.

Another object of the present invention is to makes the patient pain free without removing the native knee joint and this benefit is long lasting. Patients are pain free and have increased range of movement and mobility for a longer duration. The present invention solely relies on surgical cuts on the bones at the knee joint using custom made instruments. Hence it does not use any prosthetic implants. With no prosthetic implants used there are several advantages such as reduced chances of infection related to foreign material implantation.

The primary objective of the surgical technique is to restore the knee joint anatomy by performing partial cuts to tibia and femur. Restoring the joint space allows natural mechanism of joint movement. Making particular cuts in accordance with the present invention on to the cancellous bones of the femur and tibia restores and preserves the knee joint anatomy and may lead to cartilage regrowth mainly because of stem cells (multipotent and pluripotent) driven regeneration in their normal positions hence allowing for natural articulation and improvement in the joint movement and shock absorption.

Further the object of the present invention is more cost effective than the TKR. Also, the TKR revisions are steadily increasing due to various reasons such as mechanical failure, infection etc. which is not a factor with the present invention.

Furthermore the object of the present invention is to minimize the learning curve for surgeons while minimizing operation recovery time, blood loss, soft tissue damage, and incision length.

Further the object of the present invention is to result in a faster, less painful return to function for the patient with a relatively small scar.

### BRIEF DESCRIPTION OF THE DRAWINGS

FIG. 1A, and FIG. 1B represents anatomy of a healthy knee joint for better understanding.

FIG. 2 depicts a flexed normal knee showing different important ligaments.

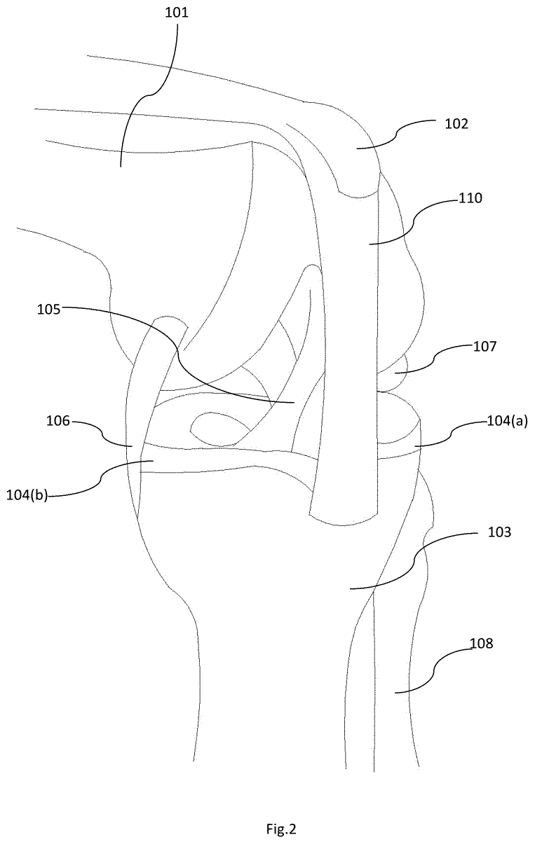

FIG. 3 shows comparison between a Normal Cartilage and a Damaged Cartilage.

FIG. 4 shows anterior view of first incision on a damaged knee in supine position in accordance with present invention.

FIG. 5 shows first two cuts on the cancellous bone using custom saws in accordance with present invention.

FIG. 6 shows damaged knee flexed in 90 degree with deepening the cuts on the cancellous bones and removal of the cuneate block in accordance with the present invention

FIG. 7 shows other steps of the method of performing the surgery in accordance with the present invention.

FIG. 8 represents a flow chart briefly explaining the steps of performing the surgery in accordance with the present invention.

FIG. 9A shows an X-Ray of the damaged arthritic knee joint of the patient

FIG. 9B shows an X-ray of the same patient after surgery showing recovery to normal anatomical position of the knee joint.

### DETAILED DESCRIPTION OF THE PRESENT INVENTION

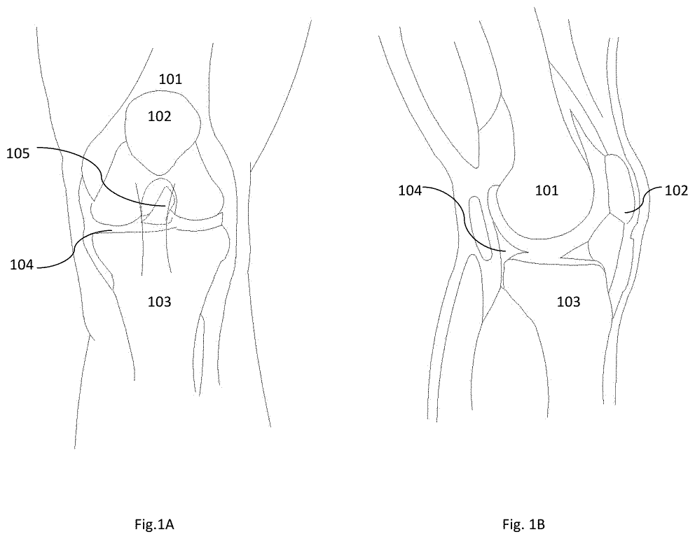

A joint generally consists of two relatively rigid bony structures that maintain a relationship with each other. Soft tissue structures spanning the bony structures hold the bony structures together and aid in defining the motion of one bony structure to the other. A normal knee anatomy is shown in FIG. 1. The point of articulation of the femur with the tibia defines a knee and structures like the distal femur (101), the proximal tibia (103), the patella (102), and the soft tissues, including ligaments, within and surrounding the knee joint. The knee is divided into three parts namely; medial (the inside part of the knee), lateral (the outside part of the knee), and patellofemoral (the joint between the kneecap and the femur). “The medial compartment comprises the medial joint surfaces of the femur (101), tibia (103), and the meniscus (104(b)) wedged there between. The lateral compartment comprises the lateral joint surfaces of the femur (101), tibia (103), and the meniscus (104(a)) wedged there between. The patellofemoral compartment comprises the joint between the undersurface of the kneecap or patella (102), patella tendon (110) and the femur (101).” Four ligaments are especially important in the stability, alignment and functioning of the knee: 1) the anterior cruciate ligament (105); 2) the posterior cruciate ligament (106); 3) the medial collateral ligament (106); and 4) the lateral collateral ligament (107).

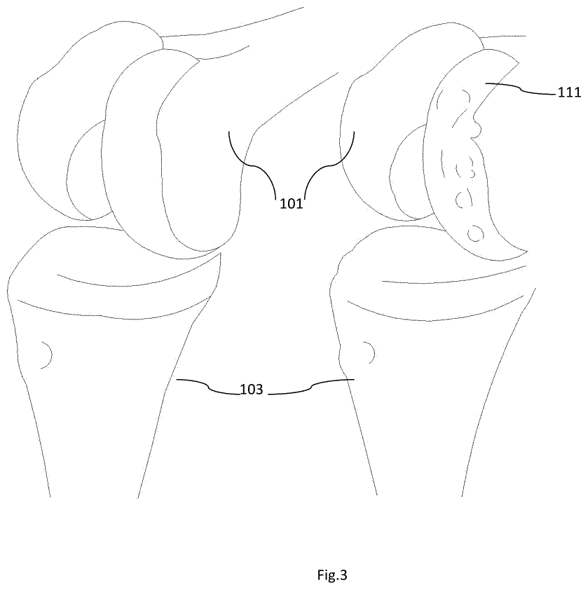

A common disorder of joints is degenerative arthritis. Degenerative arthritis causes progressive pain, swelling, and stiffness of the joints. Osteoarthritis of the knee can cause symptoms ranging from mild to disabling. As shown in FIG. 3 in an arthritic knee, protective cartilage (111) at the point of articulation of the femur (101) with the tibia (103) is often worn away, allowing the femur (101) to directly contact the tibia (103). Because of the progressive nature of the disease, many patients with osteoarthritis of the knee eventually require operative treatment.

A variety of procedures have been described for treatment of the osteoarthritic knee, including arthroscopic debridement, osteochondral or chondrocyte transplantation, high/Proximal tibial osteotomy, distal femoral osteotomy, arthroplasty, and arthrodesis. This bone-on-bone contact can cause significant pain, discomfort, and disability for a patient and will often necessitate surgery. While attempting to select the most apt surgical option for a patient longevity or durability is an important factor to consider. Thus the method of performing restoration of knee surgery in accordance with the present invention is “A natural way of treating osteoarthritis of knee to restore the knee joint to its anatomical condition.” The foundation of the present invention is to preserve and restore the knee joint as close as anatomically possible. The joint cartilage system that is responsible for joint articulation as well as shock absorption is typically damaged due to osteoarthritis. The present invention by preserving the knee anatomy allows these cartilages to regrow in their normal positions hence allowing for natural articulation and improving the joint movement and shock absorption.

Materials and Instruments in Accordance with the Present Invention:

The following instruments and materials needed for the Surgery:

Different sizes of Retractor used for the retraction of the skin like small retractor, self-holding retractor, and big retractor.

Different types of Forceps like Artery Forceps, Regular Forceps, Painting And Draping Forceps, and sequestrum Holding Forceps.

Knifes for incisions (122)

Bone lever

Custom Made Saws(123)

Nibbler

Thin osteotome and Thick osteotome

Hammer

Custom made Brace

IITV

Many patients, with different social strata and age groups with different body conditions like HTN, D.M. severely impaired renal functions due to long and continuous consumption of heavy analgesics, have undergone this surgery successfully.

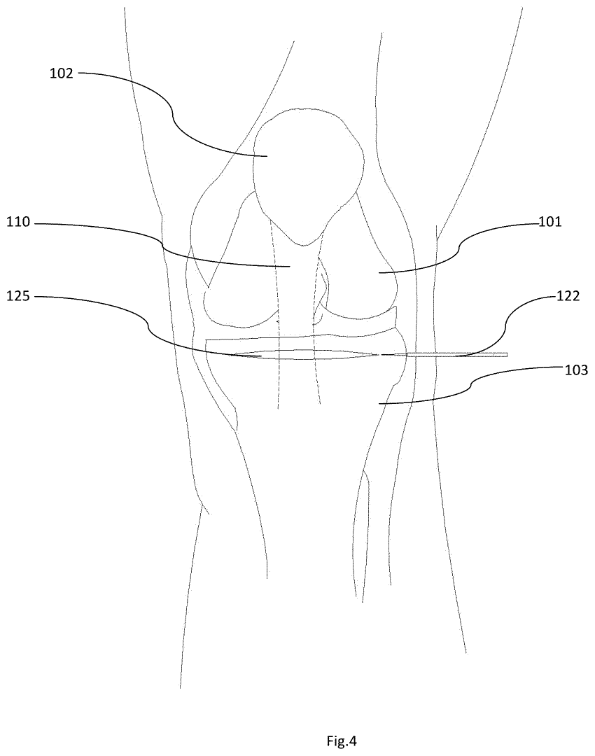

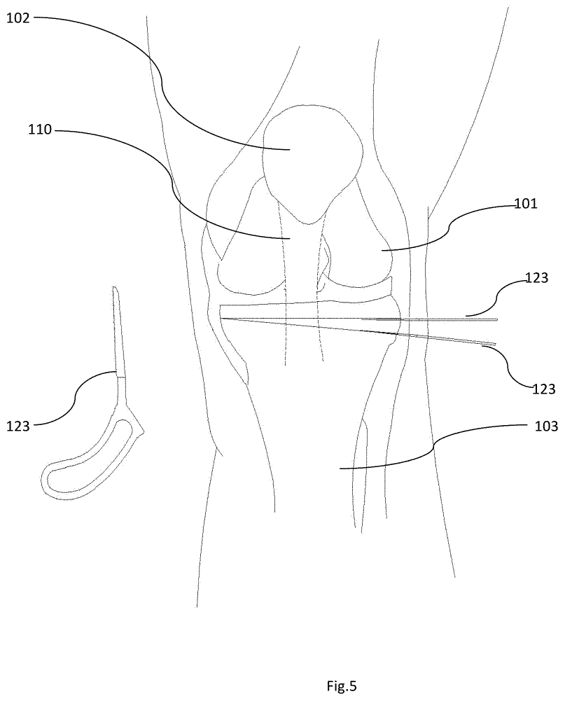

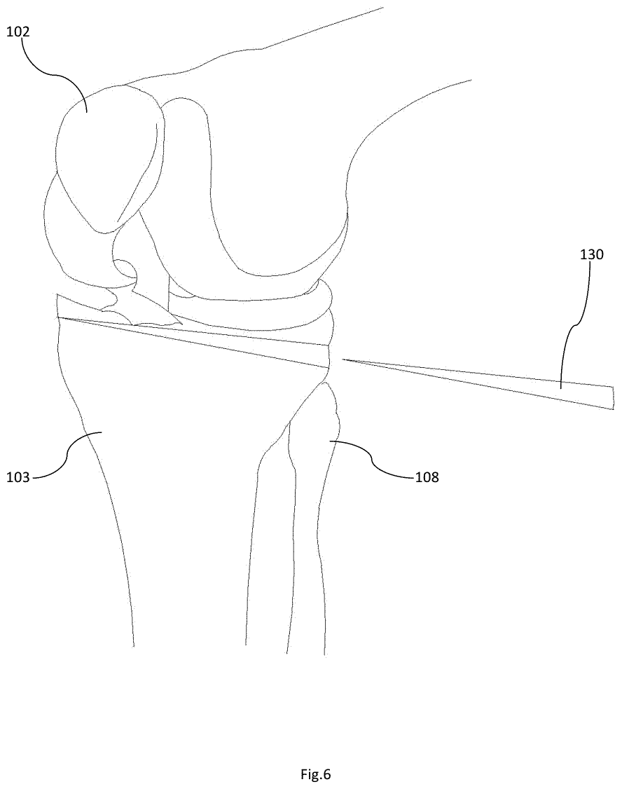

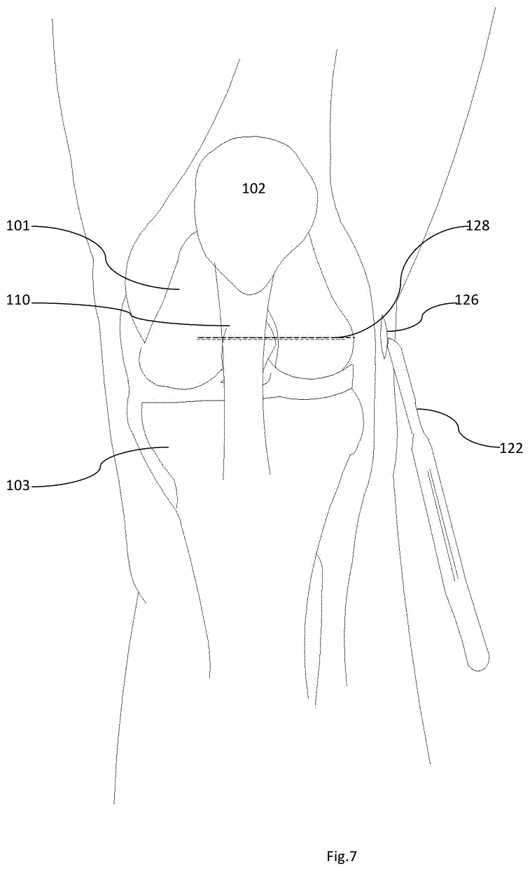

The present invention is performed under spinal anesthesia in supine position, after applying tourniquet over thigh. Referring to FIG. 4, FIG. 5, FIG. 6 and FIG. 7 one embodiment of a method of performing restoration of knee surgery in a patient in accordance with the present invention includes positioning the patient's leg, by making two separate incisions near the knee joint. In accordance with the present invention, one incision (125) is made transversely below the knee joint and then with the help of a specially designed instrument (123), appropriate cuts are put over the lateral side of the knee joint and by making sure and safeguarding the medial ligament (106), patella tendon (110) and other ligaments, lastly by making the final cut (128) on the cancellous bone of the femur to relieve the intraosseous pressure and among other final steps.

Method of performing restoration of knee surgery in accordance with the present invention is as follows.

Pre Operative Procedure:

Method of performing restoration of knee surgery in accordance with the present invention is performed under spinal anesthesia after applying pneumatic tourniquet over the lower ⅓^{rd} of thigh. Intravenous antibiotic is given as ‘loading dose’ before applying tourniquet. Routine ‘painting and draping’ is done over the knee joint to be operated keeping the leg free for manouvreing during surgery.

Operative Procedure:

Leg Position:

Method of performing restoration of knee surgery in accordance with the present invention is performed by keeping the patient in supine position with knee extended. The knee is later flexed to 90° to have clear view and deepen the cut using Thin Osteotome on the cancellous bone of the tibia.

Incision:

Thereafter, a transverse incision (125) is put directly over patellar tendon from medial ligament of the knee to lateral ligament of the knee.

Dissection:

Dissection in accordance with the present invention is carried out on either side of patellar tendon (110) to make space over upper end of tibia (103). If infrapatellar pad of fat is in abundance then part of it is removed. Thus, a clear space of about 1.5 inches is created. The medial ligament (106) of the knee joint is protected by a ‘bone lever’. Incase of excess bleeding the Artery forceps is used to rupture the blood vessels.

Cuts:

Under the guidance of IITV, using a small custom made ‘finger saw’ with a pistol handle an upper cut is made parallel to the articular surface of tibia (103) and impression of the cut is made. Keeping the saw in place, with a second saw a ‘cuneate incision’ is made and impression is created on tibia below the 1^{st} cut. Depending on the degree of deformity, accordingly the 2^{nd} cut is put. These 2 cuts meet at the apex of the ‘cuneate incision’. The saws (123) are removed one by one and the cuts are deepend using the Thin osteotome. Later both the saws and retractors are removed and only the bone lever is in place. After the impressions made knee is flexed to 90° and it is fixed with a foot rest in that position. This is very important part of the surgery because flexion of the knees allows blood vessels and nerves to fall back and thus they are well protected. At this stage, Now with a sharp osteotome slowly the cuts put by cuneate incision are deepened further. The surgeon have to make sure that the cut should not be beyond the posterior cortex of tibia. Using a Thick Osteotome the base is remove and suitable cuneate block (130) is removed, at this stage the foot rest is removed and knee is extended using opposite force. Incase, there are any fragments are left after removing the the cuneate block the artery forceps or thin osteotome can be used to clean it up. Finally the medial bone lever is removed.

Now at this stage, 3 deformities can be corrected simultaneously: 1) Fixed Flexion Deformity 2) Genu Varum 3) Internal Torsion. The position of the knee after correcting this deformity is checked under IITV. Thus in this procedure, the knee joint is not cut open.

Second Incision:

A second small vertical incision (126) about 1.5 inches is taken over the lateral aspect of femoral condyle about 1.5 inches away from articular surface of femoral condyle by cutting the tensor fascia lata. Again by putting a ‘bone lever’ over the posterior aspect of femoral condyle, vital structures are protected.

Removal of intraosseous pressure:

After confirming the level of cut under IITV, with a sharp osteotome a transverse cut (128) is made over femoral condyle upto medial cortex of femur. This is done to remove the pressure built in the femoral condyle.

In this procedure, no muscle is cut and cuts being superficial require only subcutaneous closure and skin closure using stitches and stapler. Further, padded dressing is applied and adequate pressure is given by elastocrepe bandage. After applying elastocrepe bandage, tourniquet is released and removed. Padded dressing is extended over the whole thigh upto about 2 inches below the inguinal ligament.

Post-Operative Procedure:

A customised ‘long brace’ is then applied on the operated limb which is prepared prior to surgery depending on the deformities of the knee. This brace is worn by patients for 30 days following surgery. Patient is made to walk within 24 hours post surgery with full weight bearing and patient starts ‘static quadriceps’ exercise which helps to keep the normal tone and power of the muscles. After 30 days, ‘long brace’ is removed while static quadriceps exercise is continued. Usually patients gain about 90 degree of flexion within few minutes at the end of 30 days post operatively. Later a customised ‘short brace with hinge’ which is prepared prior to surgery is applied which extends from lower half of the thigh to upper half of leg is worn for another 6 to 8 weeks. This allows knee movement. In about 2 weeks after removal of ‘long brace’, patient gains full range of movement.

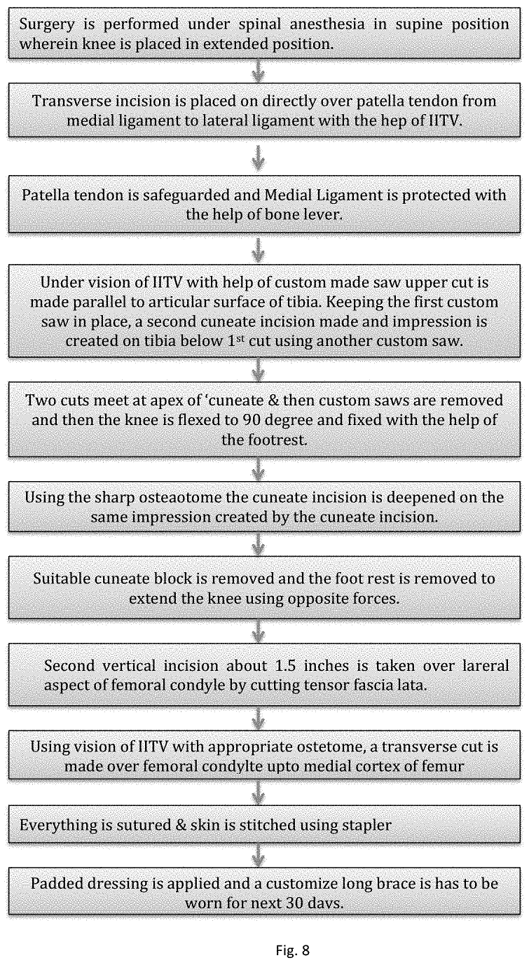

As recited and shown in FIG. 8 the method of performing restoration of knee surgery in accordance with the present invention solely relies on surgical cuts on the cancellous bones at the knee joint. Thus, there is no need of prosthetic implants and hence there are several advantages such as reduced chances of infection related to foreign material implantation and subsequent reduced requirement of pre and postoperative antibiotics. Knee replacement surgery or arthroplasty typically involves a resurfacing of the knee joint in which the femur, or thigh bone, and/or tibia, or shinbone, are covered with artificial materials such as metal or plastic thereby replacing the irregular surfaces caused by the arthritis with smooth surfaces. The undersurface of the patella, or kneecap, may also be replaced with artificial materials. The present invention makes the patient pain free without removing the native knee joint and this benefit is long lasting. Patients are pain free and have increased range of movement and mobility for a longer duration.

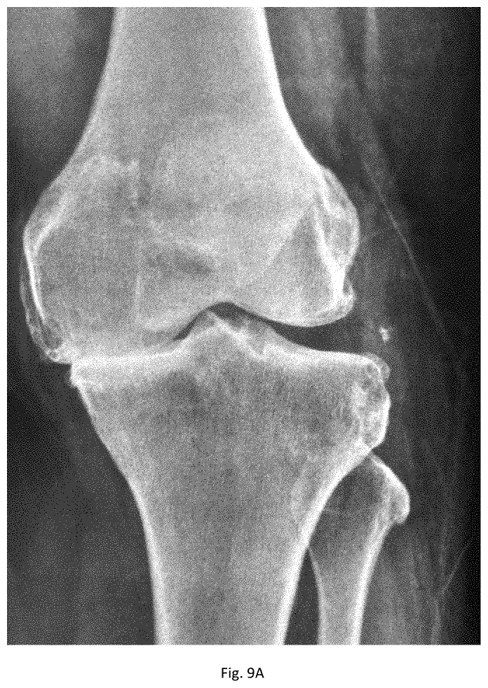

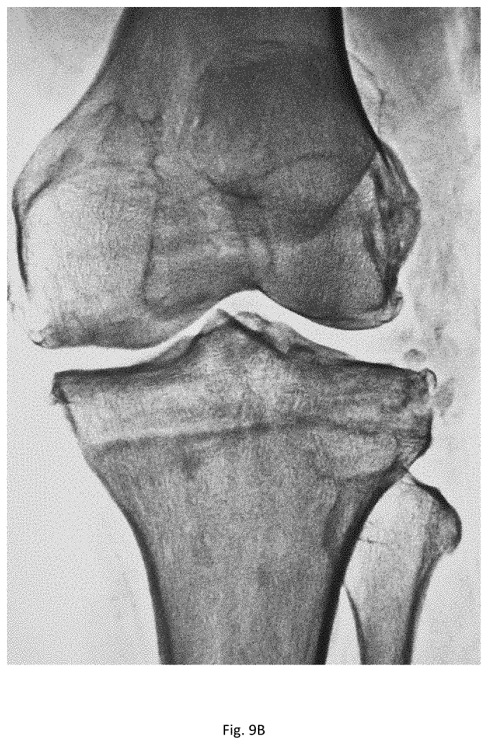

FIG. 9A and FIG. 9B are the pre operative and post operative x ray of a patient. The pre operative x-ray clearly shows the damaged arthritic knee joint of the patient. The method of performing restoration of knee surgery in accordance with the present invention is performed by learned doctor and after surgery in few weeks the X-ray of the patient is taken of the same knee. It shows considerable recovery to normal anatomy of the knee joint in that patient.

TABLE 1
Comparison chart between the present invention, TKR and HTO

| | Present Invention | Knee replacement surgery | High Tibial Osteotomy |
|---|---|---|---|
| Durability | Lasts life long | About 10-12 years | About 6-7 years |
| Chance of | Almost nil | Very high because of the use | Less than TKR, but still high |
| infection | | of implants in our body | because of implants |
| Complication | Almost nil | All natural protective | Once fails, even TKR |
| | | mechanisms are gone forever | becomes difficult |
| | | as the joint is removed. | |
| Implant | No implant, hence no | Failure because of foreign | Failure due to infections, |
| failure | failure | body reaction, infection, | injuries etc. |
| | | injury etc. | |
| Function of | All normal activities | Restricted activity | Not absolutely normal |
| knee after | Almost unrestricted | | |
| surgery | activity | | |
| Social and | Normal social and | Sports activity not possible | Certainly restricted due to |
| sports activity | sports activity is | and social activity restricted | implants |
| | allowed | | |

The principles, preferred embodiments and modes of operation of the present invention have been described in the foregoing specification. However, the invention should not be construed as limited to the particular embodiments, which have been described above. Instead, the embodiments described here should be regarded as illustrative rather than restrictive. The present invention is performed on various patients with different type of deformities. Since the concept of the surgery remains the same, there might different permutation and combination, variation performed depending on each case of the patient. Others may make variations and changes without departing from the scope of the present invention as defined by the following claims.

1. A method of performing a restoration of knee-joint surgery involving a tibia and femur on a surgical table, the method comprising:
positioning the tibia and the femur such that the knee-joint is disposed in a extended position initially;
first incising skin transversely and flesh layers proximate the knee-joint;
retracting the skin and flesh layers proximate the knee-joint to with the help of retractor;
first parallel cut on to the articular surface of tibia making an impression of the cut by keeping the custom made saw in place;
second cuneate cut made in an angle on tibia just below the first parallel cut depending on the degree of deformity;
both the saws are removed and knee is flexed to 90° to have a clear view followed by deepening the cuts using a sharp osteotome and the base is removed using a Thick Osteotome;
cuneate block is removed and cleaned for any remaining fragments;
Knee is again extended using opposite forces and checked under IITV for confirming the correction of deformity;
second lateral incision about 1.5 inches is taken over lateral aspect of femoral condyle by cutting tensor fascia lata;
again the vital structures are protected;
Third transverse cut is made on over femoral condyle upto medial cortex of femur to remove the intraosseous pressure; and
Skin closure using stitches and stapler.
With Post operative custom made brace to be worn after surgery.

2. The method as per claim 1 is carried out under spinal anesthesia and Intravenous antibiotic is given as a loading dose before applying tourniquet.

3. The incisions in the method as per claim 1 is carried under vision of IITV and made using regular surgical small knife.

4. The first incision as per claim 1 is put transversely over patellar tendon from medial ligament of the knee to lateral ligament of the knee.

5. The method as per claim 1 is carried out in such a way that the patellar tendon ligament and medial ligament are firstly safeguarded and preserved in its normal anatomy.

6. The surgical cuts as per claim 1 are made using a small finger saw with a pistol handle and deepened using a Thin and Thick Osteotome.

7. As per the method in claim 1 the first and the second cuts or the impressions made on the articular surface of tibia meets at the apex of the cuneate incision.

8. The surgical cuts as per claim 1 are made on the cancellous bones of the femur and tibia to activate the natural mechanism of the body and regenerate the stem cells in synovial membrane

9. The third transverse cut as per claim 1 is deepened upto 80% using a sharp osteotome to remove the pressure built in the femoral condyle.

11. The method as per claim 1 requires a special brace to be worn postoperative surgery for 30 days.

12. The method as per claim 1 makes the patient pain free without removing the native knee joint and preserves the natural anatomy of the knee joint.
